(19) United States
(12) Patent Application Publication
(10) Pub. No.: US 2013/0253317 A1
(43) Pub. Date: Sep. 26, 2013

(54) ULTRASOUND IMAGING SYSTEM WITH PATIENT-SPECIFIC SETTINGS

(71) Applicant: Thomas Patrice Jean Arsene Gauthier, Seattle, WA (US)

(72) Inventor: Thomas Patrice Jean Arsene Gauthier, Seattle, WA (US)

(73) Assignee: KONINKLIJKE PHILIPS ELECTRONICS N.V., EINDHOVEN (NL)

(21) Appl. No.: 13/885,873

(22) PCT Filed: Dec. 14, 2011
(86) PCT No.: PCT/IB2011/055655

§ 371 Date: May 16, 2013

Related U.S. Application Data
(60) Provisional application No. 61/423,129, filed on Dec. 15, 2010.

(51) Int. Cl. A61B 8/08; A61B 8/13; A61B 8/00

(52) U.S. Cl. CPC A61B 8/085; A61B 8/467; A61B 8/54; A61B 8/13
USPC 600/437

(57) ABSTRACT

The present invention relates to an ultrasound imaging system (10) and a corresponding method which enable that in subsequent examinations of the same patient ultrasound images are acquired under conditions such that the images can be compared and can be used to monitor disease progression. The proposed ultrasound imaging system (10) comprises an image acquisition unit (12) configured to acquire an ultrasound image based on a set of acquisition parameters, a user input (18) for entering (S10) a patient identifier (26), a database access (28) configured to access a database of sets of acquisition parameters, wherein the sets of acquisition parameters are associated with patient identifiers (26), and a control unit (16) configured to automatically retrieve (S12) a set of acquisition parameters that is associated with said patient identifier (26) based on an entered patient identifier (26) and control the image acquisition unit (12) to acquire (S22) an ultrasound image based on the retrieved set of acquisition parameters.

### FIELD OF THE INVENTION

The present invention relates to a system and method for acquiring ultrasound images. The invention also relates to a computer program for implementing said method.

### BACKGROUND OF THE INVENTION

Ultrasound imaging is commonly used to diagnose many different diseases and the progression of these diseases. In order to best adapt the image acquisition to the specific disease, the organ under investigation, and different characteristics of the patient, there are numerous acquisition parameters that need to be set appropriately. These parameters are related to the transmission and reception of the ultrasound signals, the processing of the measured signals, image reconstruction, image display and image storage. They include such operating parameters as the depth of an image, the location of the transmit focus, the number of focal zones, whether to use the B mode or color Doppler mode, whether harmonic or fundamental frequencies are to be used for imaging, image resolution, frame rate etc. In the clinical context of monitoring patient response to cancer treatment, the same ultrasound scanner is used for multiple patients and the settings are often changed. Manually changing the parameters for every acquisition is time consuming and error-prone. Therefore, many systems include tissue-specific presets (TSP). These are sets of imaging parameter values that have been optimized for a particular application, for example imaging of the liver or imaging of the carotid artery. With any given ultrasound transducer, the manufacturer typically offers a selection of tissue-specific presets that the user may choose from to quickly set up the ultrasound scanner for a particular scanning task. Often, these general presets need to be changed and adapted to specific patients.

U.S. Pat. No. 5,315,999 discloses an ultrasound imaging system in which sets of imaging parameter values are saved as preset modes. When a user later selects one of the preset modes, the system automatically operates in accordance with the corresponding set of imaging parameter values. The system can store preset modes for different exam types, for different image displays, for different patients and for different users.

The inventors realized that it is crucial to guarantee that for each patient the same imaging parameter values are used throughout the patient follow-up to ensure reproducibility of scans and allow for scans at different points in time to be compared with one another to assess patient response to treatment.

### SUMMARY OF THE INVENTION

It is an object of the present invention to provide an ultrasound imaging system that ensures that in subsequent examinations of the same patient ultrasound images are acquired under conditions such that the images can be compared and can be used to monitor disease progression.

It is a further object of the present invention to improve the workflow for ultrasound imaging in monitoring disease progression.

In a first aspect of the present invention a an ultrasound imaging system is presented that comprises

an image acquisition unit configured to acquire an ultrasound image based on a set of acquisition parameters,

a user input for entering a patient identifier,

a database access configured to access a database of sets of acquisition parameters, wherein the sets of acquisition parameters are associated with patient identifiers, and

a control unit configured to automatically retrieve a set of acquisition parameters that is associated with said patient identifier based on an entered patient identifier and control the image acquisition unit to acquire an ultrasound image based on the retrieved set of acquisition parameters.

Based on the entered patient identifier (which can be a number that uniquely identifies a patient, as it is commonly used in hospitals), the ultrasound imaging system looks up the set of acquisition parameters that are stored in the database associated with this patient identifier. The stored acquisition parameters can be either the parameters that were used for this patient at the last examination or they can be acquisition parameters that were stored specifically as “preferred” parameters that should be used for examinations of this patient.

In an embodiment according to the invention, the ultrasound imaging system further comprises a selection user interface, wherein the control unit controls the image acquisition unit to acquire an ultrasound image if the selection user interface is operated by a user.

In a preferred embodiment according to the invention, the sets of acquisition parameters are further associated with body organs, the control unit is adapted to determine the body organs for which sets of acquisition parameters are available for a given patient identifier, and the selection user interface is adapted for allowing a user to choose from among the determined body organs. If for a given patient several organs have been examined with the ultrasound imaging system, this ensures that the acquisition parameters are also adapted to the specific organ of the patient.

In another preferred embodiment of the present invention, the acquisition unit comprises an exchangeable transducer unit and the sets of acquisition parameters comprise one parameter for identifying a transducer unit. Storing information about the used transducer together with other acquisition parameters allows the ultrasound imaging system to ensure that the same transducer is used for follow-up studies of a patient.

In another embodiment of the present invention, the ultrasound imaging system further comprises an automatic selection unit configured to automatically detect a near organ that the image acquisition unit is near to. For example, the image acquisition could acquire a test image as soon as it is placed on the patient's body. Based on this test image, the automatic selection unit could determine which organ the acquired test image corresponds to. This could be done for example by comparing the test image with images that have previously been acquired from the same patient or it could be compared with a database of ultrasound images that are characteristic for certain organs.

Automatically detecting the organ from which the user is about to acquire a diagnostic image has the advantage that the workflow can be simplified even further. For example, if for a given patient sets of acquisition parameters are available for liver, heart and gall bladder, and the automatic selection unit detects that the user has placed the image acquisition unit near the liver, the control unit could automatically retrieve the set of acquisition parameters associated with the liver. Subsequently, it could control the image acquisition unit to acquire an ultrasound image based on the retrieved set of acquisition parameters associated with the liver.

In a further aspect of the present invention a method is presented that comprises

entering a patient identifier,

automatically retrieving from a database a set of acquisition parameters that is associated with said patient identifier, and

controlling an image acquisition unit to acquire an ultrasound image based on the retrieved set of acquisition parameters.

According to further aspects of the present invention a computer program for implementing said method is provided, said computer program comprising program code means for causing a computer, when said computer program is carried out on the computer, to carry out the steps of:

displaying a user input field for entering a patient identifier,

automatically retrieving from a database a set of acquisition parameters that is associated with said patient identifier, and

controlling an image acquisition unit to acquire an ultrasound image based on the retrieved set of acquisition parameters.

Preferred embodiments of the invention are defined in the dependent claims. It shall be understood that the claimed method and the claimed computer program have similar and/or identical preferred embodiments as the claimed device and as defined in the dependent claims.

### BRIEF DESCRIPTION OF THE DRAWINGS

These and other aspects of the invention will be apparent from and elucidated with reference to the embodiment(s) described hereinafter. In the following drawings

FIG. 1 shows a block diagram of an embodiment of an ultrasound imaging system according to the present invention,

FIG. 2 shows a flow chart of a method according to the present invention, and

FIGS. 3 to 5 show preferred embodiments of a user interface for an ultrasound imaging system according to the present invention.

### DETAILED DESCRIPTION OF THE INVENTION

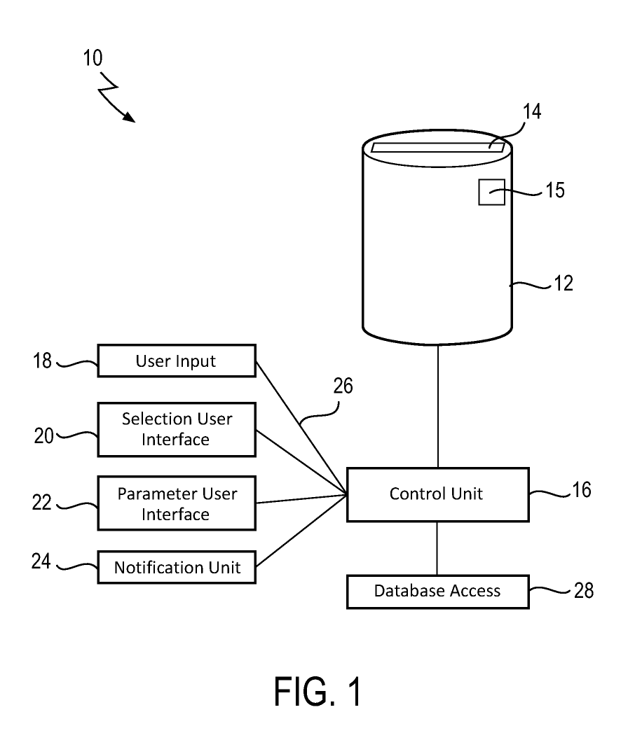

FIG. 1 shows a schematic block diagram of an ultrasound imaging system 10 according to the present invention. The image acquisition unit 12 comprises an exchangeable transducer unit 14 and a transducer sensor unit 15 which is capable of recognizing different exchangeable transducer units 14.

The control unit 16 is connected to a number of user interface components: the user input 18, the selection user interface 20, the parameter user interface 22, and the notification unit 24. Based on the patient identifier 26 that is entered in the user input 18, the control unit 16 is adapted to retrieve a set of acquisition parameters that corresponds to this patient. In this embodiment, the user input 18 directly takes a patient identifier as input, e.g. a number that is unique for this patient. In other embodiments, the user input 18 could also comprise input fields for patient name, gender, and birth date, such that the user input 18 can look up the corresponding unique patient. Once a patient has been uniquely identified, the control unit 16 uses a database access 28 to retrieve corresponding acquisition parameter settings. The database access 28 could be a database that is part of the ultrasound imaging system 10. More commonly, it is a connector to a network which includes a dedicated database server. For example, in a hospital, there is a typically a network-attached server that stores images and image acquisition settings such that they can be accessed from imaging devices in different locations.

The selection user interface 20 allows the user to choose between different organs for which acquisition parameters for this patient are available. For example, the patient could have been examined previously at the heart, the liver, and the prostate; this would be shown as options to the user. If there is only one set of acquisition parameters in the database that is associated with the patient identifier 26 of the current patient, in one embodiment of the invention (not shown), the selection user interface 20 is automatically reduced to only show a button, which allows the user to immediately start image acquisition with this set of acquisition parameters. In one embodiment of the present invention, the acquisition parameters that are retrieved by the control unit 16 from the database access 28 are shown to the user and the user is given the opportunity to change some of the parameters. Even if, in this case, the acquisition parameters are not exactly identical to the acquisition parameters that were previously used for this patient, the user is made aware of the fact that he deviates from previously used settings Likely, he would only do so if there is good reason, e.g. if it has turned out that previously used settings are disadvantageous in important aspects. The parameter user interface 22 could highlight those parameters which are different from the previously used parameters.

As described before, the control unit 16 is aware of which exchangeable transducer unit 14 is connected to the image acquisition unit 12. If sets of acquisition parameters are available for a given organ of a given patient, but the exchangeable transducer unit 14 does not correspond to the transducer unit that is identified in the set of acquisition parameters, the notification unit 24 is adapted for notifying the user. The notification unit 24 could be a small warning sign, e.g. an exclamation mark 24 that is displayed next to the description of the set of acquisition parameters for which the exchangeable transducer unit 14 does not match the parameters. In other scenarios, the notification sign could be more interfering, e.g. a larger blinking sign or a sound signal.

In one embodiment, the control unit 16 would be configured not to start acquiring ultrasound images in this situation. In another embodiment, the user would be given the option to proceed with the image acquisition. If necessary, the control unit 16 could change some of the acquisition parameters such that they are compatible with the exchangeable transducer unit 14 that is used instead of the preferred transducer unit that is identified in the acquisition parameters.

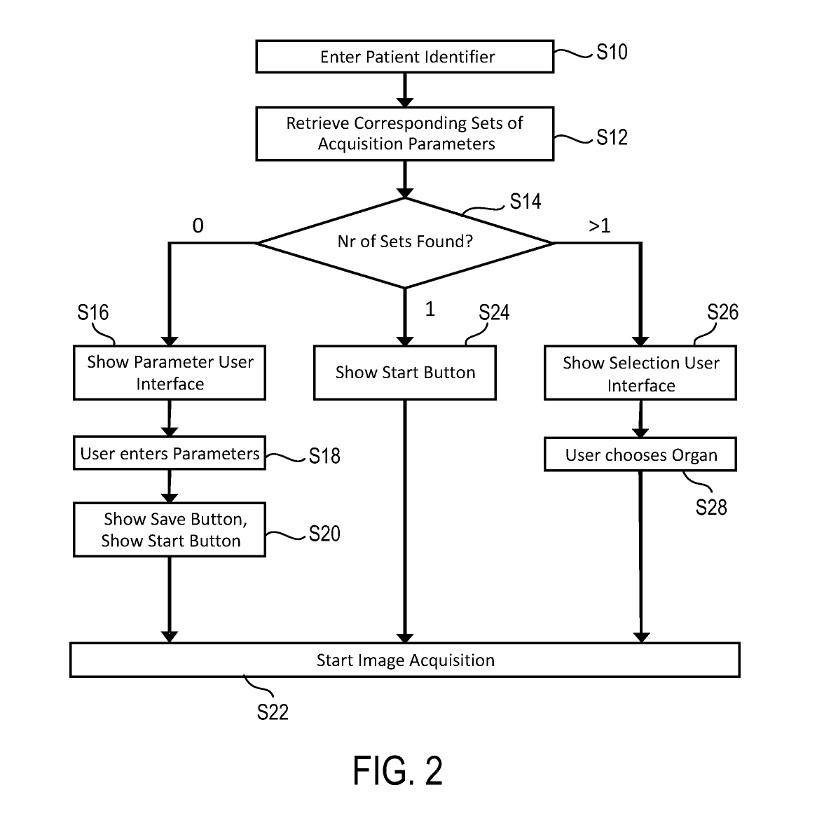

FIG. 2 shows a flowchart of one embodiment of a method according to the present invention.

In step S10, the user enters a patient identifier 26.

In step S12, the corresponding sets of acquisition parameters are retrieved from the database.

In step S14, further processing depends on the number of sets that have been found:

If no set of acquisition parameters have been found for this patient identifier 26, the parameter user interface 22 is displayed in step S16.

In step S18, the user enters a complete set of acquisition parameters (or, in an alternative embodiment, he loads a standard set of acquisition parameters).

In step S20, which occurs after a complete set of acquisition parameters has been entered or retrieved from a standard set, a Start button 34 and a Save button 38 are displayed. In another embodiment, there is a “Save and Start” button, i.e., both steps are initiated with the same user command. In yet another embodiment, there is only a Start and Save button, i.e., the user can only start the image acquisition and store the image acquisition parameters at the same time. This ensures that all acquisition parameters that are used once for an image acquisition can one be reproduced in order to obtain comparable images at a later examination.

In step S22, the ultrasound images are acquired.

If in step S14 it is determined that only one set of acquisition parameters has been found, the method proceeds to step S24 and immediately shows a Start button which allows the user to start acquiring ultrasound images with the found set of acquisition parameters. Alternatively, of course, there are also user interface elements which allow the user to change the found set of acquisition parameters or to enter a new set of acquisition parameters. For simplicity reasons, the corresponding steps are not shown in FIG. 2. In one embodiment of the invention, there is always a check whether the exchangeable transducer unit 14 that is attached to the image acquisition unit 12 matches the transducer unit that is identified in the set of acquisition parameters. This is also not shown in FIG. 2.

If in step S24 it is determined that more than one set of acquisition parameters corresponds to the entered patient identifier 26, the method proceeds to step S26 and shows the selection user interface 20.

In step S28, the user chooses one of the organs for which corresponding sets of acquisition parameters for this patient were found.

Subsequently, the ultrasound images of this organ are acquired in step S22.

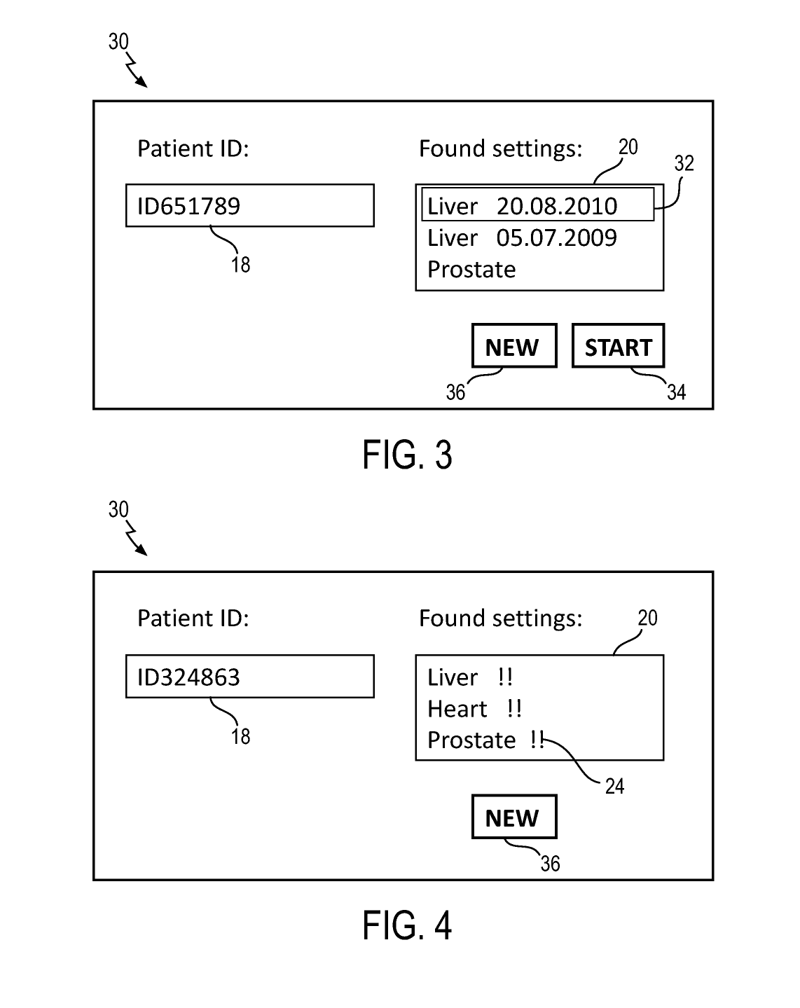

FIGS. 3 and 4 show examples of a user interface 30 of an ultrasound imaging system 10 according to the present invention. The figures show only those elements of the user interface 30 that are directly related to the present invention.

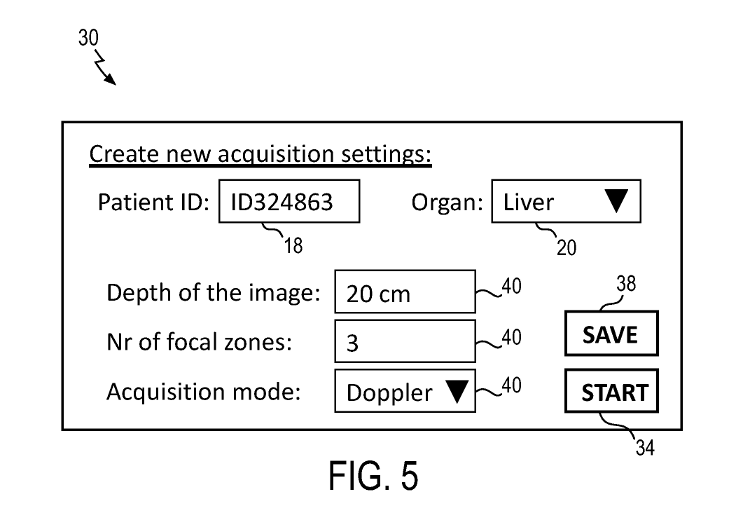

The user interface 30 comprises a user input 18 for entering a patient identifier 26. The organs for which sets of acquisition parameters for this patient are found are shown in the selection user interface 20. As shown in FIG. 3, if for one organ more than one set of acquisition parameters is found, all of the found sets of acquisition parameters can be displayed in the selection user interface 20 and differentiated by showing further information, for example the date when each of the sets of acquisition parameters was stored or when it was used for the last time. In another embodiment (not shown) the selection user interface could indicate that the sets of acquisition parameters correspond to specific (known) lesions of the patient. If an organ has several lesions, there could be different sets of acquisition parameters for the different lesions of one organ. The user can identify one of the sets of acquisition parameters for example by clicking on the corresponding line in the selection user interface 20. The selection is shown by highlighting the corresponding line 32. The user interface 30 comprises a Start button 34 and a New button 36. The start button is visible and enabled as soon as a set of acquisition parameters is found which corresponds to the given patient identifier 36 and for which a matching exchangeable transducer unit 14 has been detected by the transducer sensor unit 15. The New button is always visible and enabled and allows the user to create a new set of acquisition parameters, as shown in FIG. 5.

FIG. 4 shows an example of the user interface 30 in a scenario where for the given patient identifier 26 several corresponding sets of acquisition parameters have been found, but none of them identifies a transducer unit that matches the exchangeable transducer unit 14 that is currently attached to the image acquisition unit 12. This is indicated for each entry of the selection user interface 20 through a notification unit 24, which in this case is simply two exclamation marks. The Start button 34 is disabled and the user can not immediately start an image acquisition.

FIG. 5 shows an example of the user interface 30 that is used for entering a new set of acquisition parameters. The patient identifier 26 is entered in the user input 18, the organ, for which the set of acquisition parameters is prepared, is selected in the selection user interface 20. In this embodiment, the selection user interface allows selection an organ from a list of organs. Alternatively, the user can enter a textual description of the organ. Several parameters of the set of acquisition parameters can be entered in the parameter input fields 40. The Save button 38 and the Start button 34 are enabled as soon as a complete set of acquisition parameters has been entered. In a preferred embodiment of the invention, the Start Button 34 is implemented as a Save and Start button, i.e. the set of acquisition parameters is saved and the acquisition of the ultrasound images is started.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. An ultrasound imaging system comprising:
an image acquisition unit configured to acquire an ultrasound image based on a set of acquisition parameters,
a user input for entering a patient identifier,
a database access configured to access a database of sets of acquisition parameters, wherein the sets of acquisition parameters are associated with patient identifiers, and
a control unit configured to automatically retrieve a set of acquisition parameters that is associated with said entered patient identifier and control the image acquisition unit to acquire an ultrasound image based on the retrieved set of acquisition parameters,
wherein the sets of acquisition parameters are further associated with body organs, and
wherein the control unit is configured to determine the body organs for which sets of acquisition parameters are available for said entered patient identifier and
wherein the system further comprises a selection user interface configured for allowing a user to choose from among the determined body organs.

2. (canceled)

3. An ultrasound imaging system as in claim 1, wherein the selection user interface is operable and/or visible only if the user has entered in the user input a patient identifier for which an associated set of acquisition parameters is available.

4. (canceled)

5. (canceled)

6. (canceled)

7. An ultrasound imaging system as in claim 1, wherein the image acquisition unit comprises an exchangeable transducer unit and the sets of acquisition parameters comprise a parameter for identifying a transducer unit; and
wherein the system further comprises a notification unit configured to notify the user if the parameter for identifying a transducer unit of the retrieved set of acquisition parameters does not identify the exchangeable transducer unit.

8. (canceled)

9. An ultrasound imaging system as in claim 7, wherein the control unit controls the image acquisition unit to acquire an ultrasound image only if the parameter for identifying a transducer unit identifies the exchangeable transducer unit.

10. An ultrasound imaging system as in claim 1, further comprising an automatic selection unit configured to automatically detect a near organ that the image acquisition unit is near to.

11. A method for acquiring ultrasound images, comprising the steps of
entering a patient identifier,
automatically retrieving from a database sets of acquisition parameters that are associated with said entered patient identifier,
determining that more than one set of acquisition parameters correspond to said entered patient identifier,
showing a selection user interface,
choosing a retrieved set of acquisition parameters corresponding to a selected organ, and
controlling an image acquisition unit to acquire an ultrasound image based on the chosen retrieved set of acquisition parameters.

12. Computer program comprising program code means for causing a computer, when said computer program is carried out on the computer, to carry out the steps of
the method as claimed in claim 11.

13. An ultrasound imaging system a in claim 10, wherein the control unit controls the image acquisition unit to acquire an ultrasound image based on the retrieved set of acquisition parameters associated with detected near organ.
